(19) United States
(12) Patent Application Publication
(10) Pub. No.: US 2005/0261639 A1
(43) Pub. Date: Nov. 24, 2005

(54) Medicated ink marker

(75) Inventor: Steve A. Herweck, Nashua, NH (US)

Correspondence Address: LAHIVE & COCKFIELD, LLP., 28 STATE STREET, BOSTON, MA, 02109, US

(73) Assignee: ATRIUM MEDICAL CORP., Hudson, NH (US)

(21) Appl. No.: 10/840,181

(22) Filed: May 5, 2004

(51) Int. Cl. A61M 35/00

(52) U.S. Cl. 604/289

(57) ABSTRACT

A medicated ink marker includes a porous applicator and at least one medicated agent disposed within the porous applicator. The medicated ink marker makes no use of a reservoir from which to draw the at least on medicated agent. The at least one medicated agent is one of a non-antiseptic medicated agent and an antiseptic with an additional therapeutic function medicated agent. The porous applicator supports capillary action, such that contact of the applicator against a targeted location results in the at least one medicated agent dispensing from the porous applicator to topically apply the at least one medicated agent to the targeted location in a detectable manner without drawing additional medicated agent from another location, such as a reservoir.

### FIELD OF THE INVENTION

The present invention relates to a marker, and more specifically to a marker for use in applying an ink having an active agent. The ink is applied directly to the tissue of a patient, is detectable, and includes at least one medication, drug, and/or therapeutic agent applied to the patient for therapeutic purposes.

### BACKGROUND OF THE INVENTION

Application of a therapeutic and/or medical agent to the tissue of a patient is known in the art. In some instances, the application occurs through the coating of a medical device with an application of a medical agent for delivering medication to a patient upon usage of the medical device. For example, medical devices, such as balloons or stents, can be coated with one or more agents for controlling restenosis or smooth muscle cell hyperplasia in the human coronary arteries. The balloon or stent can have a drug eluting coating applied to one or more surfaces thereof. With this method, the drug is impregnated or made part of the coating that is applied only to the surface of the medical device structure. Known coating methods provide drug release from a bonded polymeric material or coating that surrounds one or more surfaces of the balloon or stent that generally provide a fixed rate of release of one or more medications.

Alternative to medicated devices, there are often instances where it is desirable to have a drug or agent applied directly to the tissue of a patient. In some instances, there is no need or ability to use a medical device implanted on or in the patient that includes a medicated coating for application to the tissue of the patient. For example, application directly to the skin of a patient can be done without use of a medical device because of easy access to the skin. Alternatively, some applications of medication directly to tissue during surgery may be necessary but without the option of being able to leave an implant within the patient to dispense the medication. If such an implant remains within a patient a subsequent surgery may be required to remove the implant. In other instances it may be desirable to quickly apply medication to specific locations on a patient with specificity. For example, in preparation for a surgical incision, an application of antibiotic, antiseptic, and/or anti-inflammatory agent to the specific incision location could prevent infection and inflammation in and around the surgical incision.

In still another alternative, there are instances where it is desirable to have a drug or agent applied directly to a medial device. For example, the particular drug or agent may not be easily preserved if applied to the medical device at the point of manufacture of the device. However, it may be desirous to have the drug or agent coating on at least a portion of the medical device. As such, the drug or agent can be applied directly to the medical device by the user just prior to application or implantation of the medical device.

An additional consideration is that many drugs or other therapeutic agents that are applied to the tissue of a patient, or to a medical device, are either undetectable or are otherwise not differentiable after application to the tissue. Application of a clear drug or agent can be easily missed upon subsequent inspection. Furthermore, most medications or agents are either clear or white in color, thus differentiating one medication or agent from another is nearly impossible after application to a medical device or tissue. The best way a user of a medical device can ensure that a drug or agent coated on the medical device is the desired drug or agent is if the user applies the drug or agent directly onto the medical device, or tissue, during the surgical procedure from a labeled dispenser of the drug or agent.

Application of the drug or agent directly onto the topical or internal tissue of a patient, or directly to the surface of a medical device, can be carried out using a number of different tools. For example, the drug or agent can be sprayed on, painted on using a brush, the medical device can be dipped in a liquid form of the drug or agent, or otherwise applied using an applicator. A more specific example of such an implementation involves a user dipping a brush or other tool into a reservoir of the drug or agent and then using the brush to apply the drug or agent to the surface of the tissue or medical device. The difficulty with such methods of application is that the exact dosage of drug or agent is very difficult to quantify. The amount of drug or agent collected by the tool from the reservoir of drug or agent can vary, as can the amount of drug subsequently released from the tool to the tissue or medical device. A further variation can involve the thickness of the drug or agent applied.

In some situations, the application of the drug or agent must be done quickly and efficiently, such as in emergencies or surgical procedures having very short time windows of opportunity. As such, the difficulties associated with applying a measured dosage of the drug or agent coupled with the requirement for a quick application, can make such forms of drug or agent unusable in certain situations.

### SUMMARY OF THE INVENTION

It is therefore desirable to provide an efficient and accurate device and method for applying a medicated ink marking having therapeutic or diagnostic properties directly onto the tissue of a patient or the surface of a medical device in a manner that limits the dosage of the medicated ink to prevent the application of too much of a drug or agent. The present invention provides solutions that address this need, in addition to others, as described.

In accordance with one embodiment of the present invention, a medicated ink marker includes a porous applicator; and at least one medicated agent disposed within the porous applicator. The at least one medicated agent is one of a non-antiseptic medicated agent and an antiseptic with an additional therapeutic function medicated agent. The porous applicator supports capillary action, such that contact of the applicator against a targeted location results in the at least one medicated agent dispensing from the porous applicator to topically apply the at least one medicated agent to the targeted location in a detectable manner without drawing additional medicated agent from another location.

In accordance with aspects of the present invention, the medicated ink marker further includes a holder for holding the porous applicator. The holder has a coupling for receiving the porous applicator. The holder can be an elongate structure. The porous applicator can be removably and replaceably couplable with the holder. The porous applicator can couple with the holder using a wick holder. The holder can be a casing that does not contain a medicated ink reservoir. The holder can serve as a grip for a user to grasp to control the porous applicator and application of the medicated agent to the targeted location.

In accordance with further aspects of the present invention, an identifier can be placed on the holder to convey information concerning the at least one medicated agent. The identifier can be at least one of color coded, text-based, symbol-based, and code-based.

In accordance with further aspects of the present invention, the porous applicator can be a wick.

In accordance with further aspects of the present invention, a removable cap can be placed over the porous applicator to hinder evaporation of the medicated agent. The removable cap can be replaceable.

In accordance with further aspects of the present invention, an identifier can be placed on the porous applicator to convey information concerning the at least one medicated agent.

In accordance with one embodiment of the present invention, a method of applying at least one medicated agent to a targeted location includes providing a porous applicator containing at least one medicated agent, wherein the porous applicator supports capillary action and wherein the at least one medicated agent is one of non-antiseptic and antiseptic with an additional therapeutic function. A user topically applies the at least one medicated agent to the targeted location in a detectable manner by directing the porous applicator to contact the targeted location dispensing the at least one medicated agent from the porous applicator to topically apply the at least one medicated agent to the targeted location without drawing additional medicated agent from another location.

In accordance with aspects of the present invention, the method can further include coupling the porous applicator to a holder, such that the user can control the porous applicator for application of the at least one medicated agent. The holder can be an elongate structure. The porous applicator can be removably and replaceably couplable with the holder. The porous applicator can couple with the holder using a wick holder. The holder can be a casing that does not contain a medicated ink reservoir. The holder can serve as a grip for a user to grasp to control the porous applicator and application of the medicated agent to the targeted location.

In accordance with further aspects of the present invention, an identifier can be placed on the holder to convey information concerning the at least one medicated agent. The identifier can be at least one of color coded, text-based, symbol-based, and code-based.

### BRIEF DESCRIPTION OF THE DRAWINGS

The invention will be more fully understood from the following detailed description taken in conjunction with the accompanying drawings, in which:

FIGS. 1A and 1B are diagrammatic illustrations of a marking as applied to a tissue location on a patient, in accordance with aspects of the present invention;

FIGS. 1C and 1D are diagrammatic illustrations of a marking as applied to a tissue location on a patient subsequent to application of a preparatory substance or coating, in accordance with aspects of the present invention;

FIGS. 2A, 2B, and 2C are diagrammatic illustrations of markings applied in different configurations or patterns, in accordance with aspects of the present invention;

FIG. 3 is a diagrammatic illustration of a marking applied around a target area for a surgical incision, in accordance with aspects of the present invention;

FIG. 4 is a diagrammatic illustration of a marking applied around pre-existing wound, in accordance with aspects of the present invention;

FIG. 5 is a diagrammatic illustration of a marking applied as a stamp or decal, in accordance with aspects of the present invention;

FIGS. 6A, 6B, and 6C are diagrammatic illustrations if ink markings applied in different colors, in accordance with aspects of the present invention;

FIGS. 7A, 7B, and 7C are illustrations of different medicated ink marker configurations, in accordance with embodiments of the present invention;

FIG. 8 is a perspective illustration of a holder for holding the medicated ink marker, in accordance with aspects of the present invention;

FIGS. 9A and 9B are perspective illustrations of a cap for covering the medicated ink marker for storage purposes, in accordance with embodiments of the present invention; and

FIG. 10 is a flow chart illustrating one example method of applying a marking to a surface, in accordance with one embodiment of the present invention.

### DETAILED DESCRIPTION

An illustrative embodiment of the present invention generally relates to improving the dosing and flexibility of applying different medications, drugs, therapeutic and/or other agents directly to the tissue of a patient, or to the surface of a medical device in the form of a marking. The present invention provides a clinical user with the opportunity to apply and confirm a dosage amount of a drug or agent applied in the form of a liquid, such as an ink, to create the marking. By use of an application device, the user can actually apply and control the amount of ink, and thus agent, marked on to the patient or medical device. The applicator contains a predetermined dosage or amount of drug or agent, without drawing from a reservoir of drug or agent, and the applicator ceases to emit the drug or agent when the appropriate dosage has been dispensed. As such, the clinical user cannot mistakenly apply a larger dosage than is indicated on the applicator.

The term “markings” as utilized herein is intended to relate to the result of the application of a substance containing a medication, drug, therapeutic agent, adhesive or bonding agent, and/or other agent. The substance can include a form of liquid, ink, or the like, that can be detected by a user with and/or without aid of a device after application. The resulting marking has at least some form of therapeutic or diagnostic benefit to a patient.

The terms “medication” or “medicated” as utilized herein are intended to relate to a substance or use of a substance containing or embodying a drug, agent, therapeutic agent, adhesive or bonding agent, and/or other agent having medicinal or therapeutic benefits.

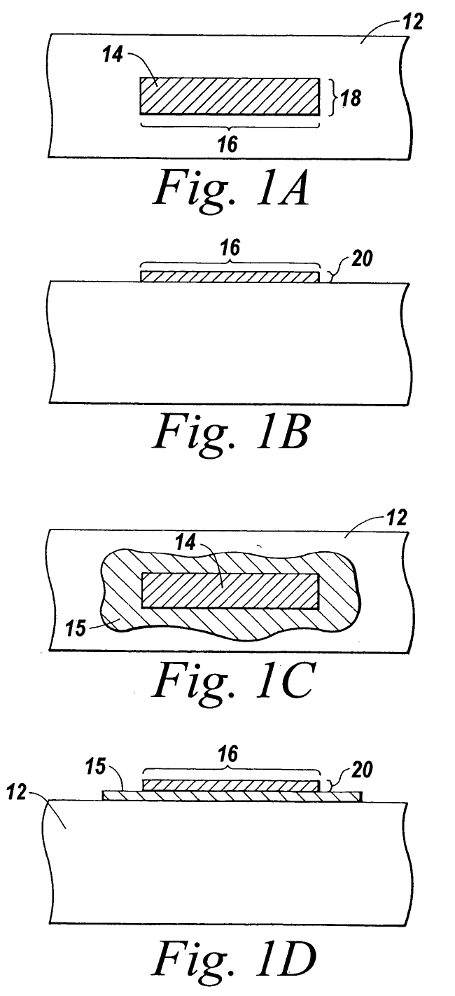

FIGS. 1A through 10, wherein like parts are designated by like reference numerals throughout, illustrate example embodiments of a medicated ink marker, according to the present invention. Although the present invention will be described with reference to the example embodiments illustrated in the figures, it should be understood that many alternative forms can embody the present invention. One of ordinary skill in the art will additionally appreciate different ways to alter the parameters of the embodiments disclosed, such as the size, shape, or type of elements or materials, in a manner still in keeping with the spirit and scope of the present invention.

The teachings of the present invention are applicable both to temporary and permanent markings. A temporarily-placed marking is defined as being a marking that can be removed or will degrade, dissolve, or otherwise dissipate at the conclusion of the therapeutic or diagnostic purpose. A permanently-placed marking, in contrast, stays within the body, or on the surface to which it is applied, for an extended period of time, or in perpetuity.

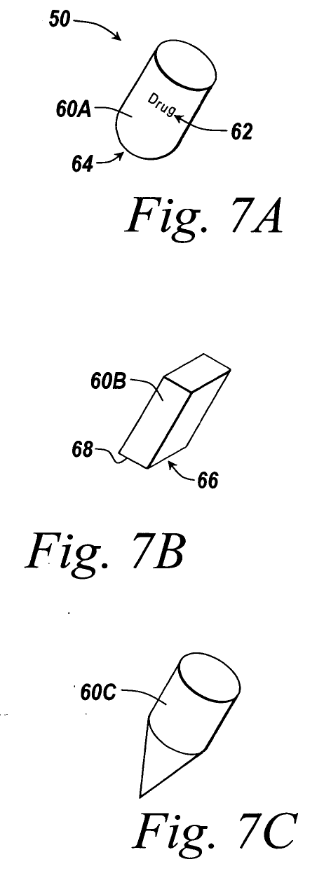

Prior to discussing the medicated ink marker of the present invention, several examples are offered of different types of markings that can be formed by use of a medicated ink marker 60 (see FIGS. 7A, 7B, and 7C).

FIGS. 1A and 1B illustrate examples wherein a marking is applied to a patient or medical device. FIGS. 1A and 1B show a marking 14 that has been applied to a surface 12, such as tissue of a patient, or portion of a medical device. The marking 14 is made by applying an ink that includes an ink carrier component, an agent component, and optionally an adhesive or bonding agent for extended or permanent ink adhesion to the surface 12. Medication saturation, loading, and dimensions of the marking 14 control the dosage of the agent that is delivered to the patient, and ultimately a fixed amount of medication is provided in the medicated ink marker 60, that provides an upper limit of medication that can be applied. The marking 14 can be made visible, or alternately detectable, by accessory device means, so that the user can confirm the application and the appropriate dosage applied to the surface 12. The marking 14 may be visible, for example, to the naked eye, or under illumination by selected types of light. The dosage of available medication or other agent can also be visibly identified by color or by combination with the dimensions and/or light refraction of the marking 14.

The marking 14 can be applied to the surface 12 in various shapes and forms. FIGS. 1A and 1B show examples where the marking 14 is applied to the surface 12. The marking 14 results from an application that includes an agent component. In one embodiment, the amount of agent in the marking 14 corresponds to the dimensional volume of the marking 14. The dimensional volume of marking applied in FIGS. 1A and 1B is equal to the product of length 16, width 18, and height 20 of the marking 14. The amount of agent on the surface 12 may thus be controlled by varying the dimensions of the marking 14. For example, the amount may be varied by varying the length 16 of the marking 14, the width 18 of the marking 14, or the height 20 (i.e., thickness) of the marking 14. The marking 14 can further be printed in a geometric shape, geometric code, universal bar code, or other format for identification and detection of the agent applied onto the surface 12. As shown in FIGS. 1C and 1D, the amount of the marking 14 deposited can further be increased by altering the surface 12 chemically or otherwise, to alter the ability of the marking to adhere to the surface 12. For example, the surface 12 can have a preparatory layer or coating 15 of a substance that improves absorption of the agent in the marking 14 by the surface 12. The layer or coating 15 can have a number of other results, such as enabling the marking 14 to better adhere to the surface 12, or to react with the marking 14 upon application of the marking 14 to the surface 12. The layer or coating 15 can be applied immediately before application of the marking 14, or can be applied at periods of time substantially before application of the marking 14 to have a more extensive effect on the surface 12.

The surface area of the marking 14 can also affect the rate of delivery of the agent to the patient. In general, a larger surface area results in a higher rate of delivery of the agent than a smaller surface area (given a same concentration of agent). Further, an irregular surface topography, including pores, may either increase or decrease the amount of marking applied to the surface 12. Hence, a clinical user may wish to consider both the volume and surface area when marking the surface 12.

More specifically, the markings 14 can have different lengths and thicknesses chosen for delivery of the appropriate dosages of the medical agents. In other words, given a uniform number of application layers, increased lengths of markings 14 result in increased dosages of the agents. Therefore, upon quick visual inspection, a user can determine and/or confirm the dosage amount provided. If the thickness is varied, the same length of marking 14 can also result in different dosages. Again, the upper limit of the dosage is mandated by the total amount of drug or agent contained within the medicated ink marker 60, because there is no reservoir or other source that can be re-visited by the user for additional medication.

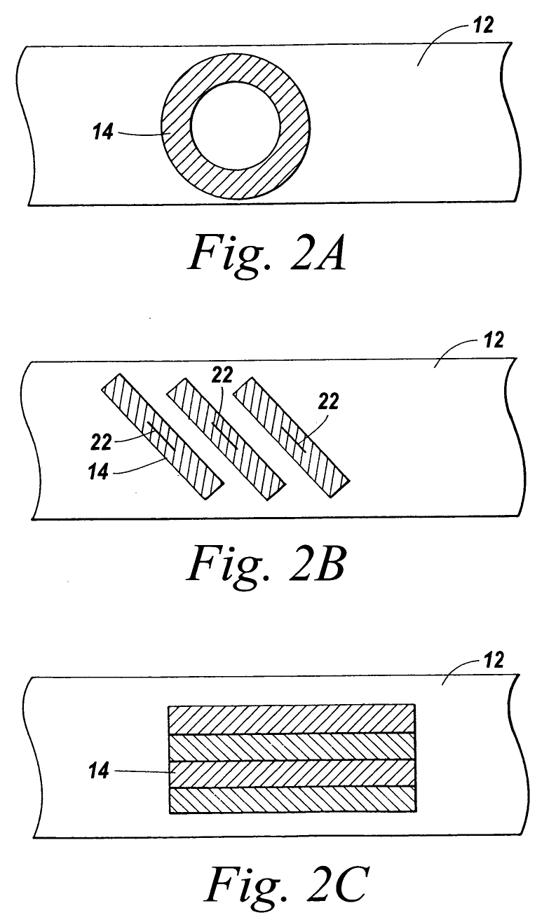

As previously mentioned, the marking 14 can be applied to the surface 12 in various shapes and forms. FIGS. 2A, 2B, and 2C show examples where the marking 14 is applied to the surface 12. The marking 14, as applied by a clinical user, can have an essentially infinite number of patterns or designs. FIG. 2A shows the marking 14 in a generally circular shape. The circle can be hollow, as shown, or solid. The circle can be placed on the surface 12 in a manner that surrounds a wound or other identifiable area on the surface 12 requiring treatment. The marking can also be placed on top of such an area.

FIG. 2B shows an additional example of the marking 14 in a pattern of angled lines. The lines are disposed over a medical fastening device 22, such as stitches or a staple. The illustration represents the use of the marking 14 as, for example, an anti-inflammatory, anti-microbial, or anti-infective agent place over the medical fastening device 22 to prevent infection. Either before, or after, insertion of the medical fastening device 22, the markings 14 are placed on the surface 12 in the approximate location of the medical fastening device 22. The agents contained within the marking 14 can be varied for the particular application. Those agents listed relative to FIG. 2B are merely illustrative of example agents or medications.

FIG. 2C shows an example of the marking 14 formed of a series of parallel lines. The parallel lines can be formed of the same ink with the same agent or agents. As shown, the lines are formed of at least two different inks and agents. This illustration shows how multiple inks and agents can form the marking 14 as applied to the surface 12. With different inks, and more particularly different agents, multiple symptoms or maladies can be treated simultaneously. The different inks and agents can form the markings 14 in whatever combination the clinical user desires, to achieve whatever therapeutic effect attributable to the particular agents being applied in the markings 14.

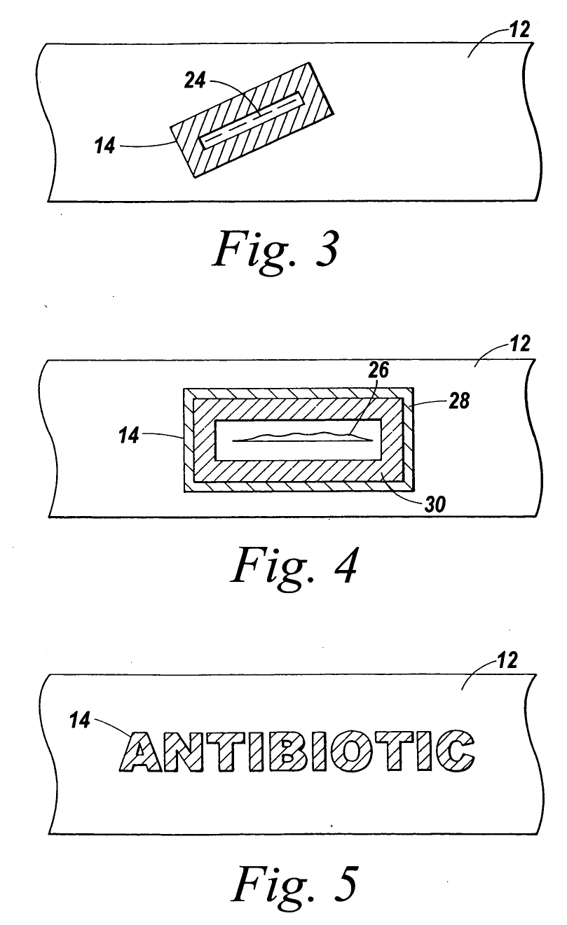

FIG. 3 shows the marking 14 in the general shape of a hollow rectangle. Inside the hollow rectangle shape of the marking 14, a dotted line 24 indicates the location of a future surgical incision. The marking 14 in such an instance can contain a therapeutic agent, such as a sterilization, anti-inflammatory, anti-microbial, and/or anti-infective agent, or some other agent as understood by one of ordinary skill in the art. The marking 14 can both serve to reduce the likelihood of infection of the pending incision, and also serve to help the surgeon visibly identify the location for making the incision. If desired, the marking 14 can be made in such a way as to indicate the desired direction, depth, or other characteristics of the pending incision.

FIG. 4 shows the marking 14 again in the general shape of a hollow rectangle. However, in the example embodiment shown, the marking 14 surrounds an existing incision or wound 26 on the surface 12 of the patient. If the marking 14 is not present prior to the incision or wound 26 as described in FIG. 3, the marking 14 can be made after the existence of the wound 26 for therapeutic purposes. The marking 14 of FIG. 4 additionally demonstrates an example embodiment wherein the marking 14 is made of two different markings containing two different agents. A first marking 28 and a second marking 30 surround the incision or wound 26. As depicted, the first marking 28 and second marking 30 can be applied in two different arrangements. The first marking 28 can serve as a border that surrounds the second marking 30. In this instance, the agent(s) in the second marking 30 are closer to the incision or wound 26, and thus have a more immediate effect, while the agent(s) in the first marking 28 are more removed from the incision or wound 26, thus having a secondary or delayed effect. Alternatively, the first marking 28 can be applied to the surface 12 and then the second marking 30 can be applied directly on top of the first marking 28 to form a layered effect. In such an instance, the agent(s) in the first marking 28 are closest to the surface 12 and the incision or wound 26, thus having a primary effect on the tissue. The agent(s) in the second marking 30 must either wait for the first marking 14 to be absorbed by the surface 12, or pass through the first marking 28 to reach the surface 12. Thus, the agent(s) in the second marking 30 have a secondary effect on the surface 12.

One of ordinary skill in the art will appreciate that there can be any number of layers as shown in FIG. 4 having the same dimensions or different dimensions as applied to the surface 12. The different layers can contain the same or different agents. For example, to increase the dosage of a particular agent in a specified location on the surface 12, multiple layers of markings 14 can be made over the specified location. Each layer is an added dosage amount. Alternatively, different agents can exist in each layer. Thus, for example, an agent that improves tissue absorption can form the first layer or first marking 28, and the therapeutic agent can exist in the second layer or second marking 30 applied on top of the first marking 28. Alternatively, two or more components of a therapeutic agent can be applied in separate markings. For example, the first marking 28 can include a first component of a therapeutic agent, while the second marking 30 can include a second component of the therapeutic agent. Once the second marking 30 is applied over the first marking 28, each of the components combines to form the therapeutic agent formed on the surface 12 for the desired therapeutic effect. In addition, the application of the layers can be staggered. For example, the first marking 28 can be applied including a therapeutic agent that has a therapeutic effect on the surface 12. After a selected period, the second marking 30 is then applied, resulting in an additional therapeutic effect. Such a process can continue as desired with additional layers of markings.

FIG. 5 shows another example embodiment of the marking 14. In this instance, the marking 14 is in a predetermined form, symbol, or word. As shown, the marking 14 is in the form of the word “antibiotic”, which would indicate that the marking 14 includes at least one antibiotic agent. The marking 14 in this instance can be applied by the user writing the desired word using the medicated ink marker 60. One of ordinary skill in the art will appreciate that the form, symbol, word, and the like, can take many different forms and can convey information as desired.

The present invention enables a physician to apply the marking 14 at a desired location on the surface 12 of a patient or medical device. For example, a user can apply antibiotic, analgesic, or anti-inflammatory medicated ink marks on a specific location where the medicated ink marks will provide the most therapeutic benefit. Further, a user can also apply a medicated ink mark to the specific desired location of dialysis needles, dialysis catheters, orthopedic implant or traction pins, laparoscopic devices, or spinal tap needles with detectable confirmation and/or visual confirmation prior to or during medical device usage.

A combination or mixture of a non-medicated ink or other substance with the ink containing the agent to form a blended ink is another method for controlling the rate of delivery of the agent to the patient. With the addition of the non-medicated ink or substance, the amount and rate of activation and/or release of the agent can be made different for different agents and/or different anatomical locations. A second non-medicated ink can further be applied as the second marking 30 to modulate the activation and/or release of the agent from the first marking 28. In addition, the surface 12 can be pre-treated with a medicated or non-medicated substance to affect absorption by the tissue.

Numerous modifications to marking shape, including pattern and orientation, will be apparent to those skilled in the art in view of the foregoing description. Accordingly, this description is to be construed merely as illustrative of the inventive concept herein. The description and illustrations should not be construed as limiting the invention.

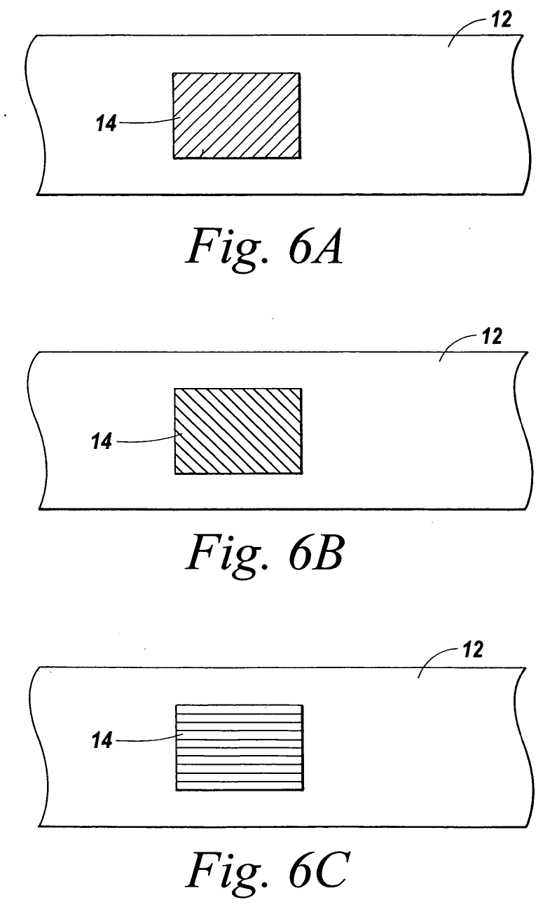

FIGS. 6A, 6B, and 6C illustrate three different embodiments of the marking 14, in the form of three different colors. FIG. 6A shows the marking 14 having a first color. FIG. 6B shows the marking having a second color. FIG. 6C shows the marking having a third color. The marking 14 is shown in the same generally rectangular shape, however, the shape of the marking 14 can vary regardless of the color.

Those skilled in the art will appreciate that a number of different bio-erodable, soluble, or permanent marker inks may be used to create the marking 14. In general, inks are formulated using a pigment to impart color, a resin binder to form the finished ink and carry the pigment, drug exuding medication, or chemical and/or solvent required to enable the binder-pigment mixture to be adhered to the tissue. Suitable pigments include but are not limited to those approved by the USFDA for medical use as listed in Title 21, Sections 73 and 74 of the Code of Federal Regulations (CFR). The following are directly applicable to tissue:

| | |
|---|---|
| Ultramarine blue | FD&C Blue |
| Iron oxide | FD&C Green |
| Titanium oxide | FD&C Red |
| Chromium-cobalt-aluminum oxide | FD&C Yellow |
| Ferric ammonium citrate | D&C Orange |
| Chromium oxide green | D&C Brown |
| Logwood extract | D&C Violet |
| Phthalocyanine green | |

In addition, those of ordinary skill in the art will appreciate that the colors can provide an indication of agent brand name, or an indication of type of agent, associated with the marking 14, as a confirmation of information conveyed by a label 62 (see FIG. 7A) of the medicated ink marker 60. For example, if a particular drug has a unique color associated with its identification or trademark, the same color can be replicated in the ink of the marking 14, such that the marking 14 is easily identified as containing that particular drug or agent. Alternatively, the color of the marking 14 can provide an indication of a type of agent found in the marking 14 applied. The use of different colors allows a physician, or other clinical user, to visibly identify the class of medication applied to the surface 12. The different color schemes for different classification types of medication provide the user with the ability to check and confirm prior to incision or other action, which medication or therapeutic application is incorporated into the ink applied to the surface 12. The specific color scheme utilized can be standardized by, for example, a national standardizing entity. The color scheme can include solid colors, as shown in FIGS. 6A through 6C, or can include simple patterns of alternating or otherwise differing colors. One of ordinary skill will appreciate the virtually infinite variability of colors, hue, fluorescence, and simple color patterns that can be used to identify particular classes or types of drugs. The colors can identify specific brand names of drugs, or any other desired clinically related attribute, as well.

As previously indicated, medical agents may be added directly to ink formulations to provide the marking 14 with medical properties. Additives and drug carrying nano-particles or microspheres containing medical agents may also be included in the ink formulation to achieve specific rates of medication permeation to local tissue. For example, fast soluble and slow soluble nano-particles or microspheres, organic solvents, and surfactants may be used to achieve a desired ink viscosity to apply the ink onto the surface 12. The solvent and surfactant are optionally removed in a subsequent process step. Other additives can include plasticizers, bio-erodable components, dye components, adhesives, bonding agents, medication stabilizers, coated and non-coated medical agent nano-particles, or microspheres, designed to improve the ink's flexibility, flow, pigment stability, shelf-life stability, and rate of surface activation and/or release into tissue or body fluid. Inks can also be formulated containing liposomes, with medication enclosed in liposomes, or phospholipid coatings. These inks can be triggered to release active compounds using an internal or external stimulus, such as ultrasound, radiation, magnetic field, or temperature, and can also be cured with application of light, such as UV light. Light, such as UV light, can also be utilized to activate the drug or agent by enhancing the application, absorbancy or adhesion of the therapeutic agent or drug.

The following examples illustrate exemplary embodiments of the present invention. A first example involves the use of the present invention in surgery. In particular, a user can make use of a visually detectable marking 14 in orthopedic surgery. In such a surgical procedure, it is often the case that there is a significant amount of blood or other fluids in the vicinity of the procedure. The user can apply the marking 14, and because it can be made with an ink that is highly visually detectable, the user can see where the therapeutic has been applied.

Another application involves laparoscopic surgery, whereby internal tissue visualization and surgical intervention is done solely by video camera and port sealed instrumentation. A laparoscope is placed through a small incision or opening in the patient. The video image is then transmitted back to a video monitor so the surgeon can see where the laparoscope is within the patient. Use of a visually detectable medicated or therapeutic ink by a suitable laparoscopic surgical instrument to form a marking 14 on the surface 12 internal to the patient facilitates application control and confirmation of therapeutic delivery to the targeted location.

Still another application of the present invention involves the use of radiopaque or otherwise machine detectable ink. In such an instance, the stability or migration of the therapeutic agent applied to a specific targeted location can be confirmed non-invasively by ultrasound, x-ray, MRI, CAT, PET, and the like. For example, the ink can be applied to a specific location during a surgical procedure. Hours or days later, the stability of the ink, or the migration of the ink, can be verified by remote monitoring because of the machine detectable qualities of the ink.

Those skilled in the art will appreciate that a number of different medical agents may be used in the marking 14. For example, anesthetic, anti-infective, lipid lowering, absorption enhancing, anti-oxidant, anti-platelet, cytostatic or cytotoxic medications can be used. In addition, medical agents that promote hollow fluid organ vaso dilation, vaso constriction, occlusion, or thrombosis can be used. The medical agents may include drugs that promote anti-thrombotic activity or can be a clot lysing agent known as a thrombolytic. The medical agents can be kinases or enzymes. The medical agents can be those that promote anti-inflammatory activity or those that promote or stimulate new bone growth. The medical agents can further include agents that promote new cell growth and/or tissue regeneration. The table below (Table #1) summarizes some examples of suitable therapeutic medical agents listed by class.

TABLE #1

| CLASS | EXAMPLES |
|---|---|
| Antioxidants | Alpha-tocopherol, lazaroid, probucol, phenolic antioxidant, |
| | resveretrol, AGI-1067, vitamin E |
| Antihypertensive Agents | Diltiazem, nifedipine, verapamil |
| Antiinflammatory Agents | Glucocorticoids, NSAIDS, ibuprofen, acetaminophen, |
| | hydrocortizone acetate, hydrocortizone sodium phosphate |
| Growth Factor | Angiopeptin, trapidil, suramin |
| Antagonists | |
| Antiplatelet Agents | Aspirin, dipyridamole, ticlopidine, clopidogrel, GP IIb/IIIa |
| | inhibitors, abcximab |
| Anticoagulant Agents | Bivalirudin, heparin (low molecular weight and |
| | unfractionated), wafarin, hirudin, enoxaparin, citrate |
| Thrombolytic Agents | Alteplase, reteplase, streptase, urokinase, TPA, citrate |
| Drugs to Alter Lipid | Fluvastatin, colestipol, lovastatin, atorvastatin, amlopidine |
| Metabolism (e.g. statins) | |
| ACE Inhibitors | Elanapril, fosinopril, cilazapril |
| Antihypertensive Agents | Prazosin, doxazosin |
| Antiproliferatives and | Cyclosporine, cochicine, mitomycin C, sirolimus |
| Antineoplastics | microphenonol acid, rapamycin, everolimus, tacrolimus, |
| | paclitaxel, estradiol, dexamethasone, methatrexate, |
| | cilastozol, prednisone, cyclosporine, doxorubicin, |
| | ranpirnas, troglitzon, valsarten, pemirolast |
| Tissue growth stimulants | Bone morphogeneic protein, fibroblast growth factor |
| Gasses | Nitric oxide, super oxygenated O2 |
| Promotion of hollow | Alcohol, surgical sealant polymers, polyvinyl particles, 2- |
| organ occlusion or | octyl cyanoacrylate, hydrogels, collagen, liposomes |
| thrombosis | |
| Functional Protein/Factor | Insulin, human growth hormone, estrogen, nitric oxide |
| delivery | |
| Second messenger | Protein kinase inhibitors |
| targeting | |
| Angiogenic | Angiopoetin, VEGF |
| Anti-Angiogenic | Endostatin |
| Inhibitation of Protein | Halofuginone |
| Synthesis | |
| Antiinfective Agents | Penicillin, gentamycin, adriamycin, cefazolin, amikacin, |
| | ceftazidime, tobramycin, levofloxacin, silver, copper, |
| | hydroxyapatite, vancomycin, ciprofloxacin, rifampin, |
| | mupirocin, RIP, kanamycin, brominated furonone, algae |
| | byproducts, bacitracin, oxacillin, nafcillin, floxacillin, |
| | clindamycin, cephradin, neomycin, methicillin, |
| | oxytetracycline hydrochloride, Selenium. |
| Gene Delivery | Genes for nitric oxide synthase, human growth hormone, |
| | antisense oligonucleotides |
| Local Tissue perfusion | Alcohol, H2O, saline, fish oils, vegetable oils, liposomes |
| Nitric oxide Donative | NCX 4016 - nitric oxide donative derivative of aspirin, |
| Derivatives | SNAP |
| Gases | Nitric oxide, super oxygenated O₂ compound solutions. |
| Imaging Agents | Halogenated xanthenes, diatrizoate meglumine, diatrizoate |
| | sodium |
| Anesthetic Agents | Lidocaine, benzocaine |
| Descaling Agents | Nitric acid, acetic acid, hypochlorite |
| Chemotherapeutic Agents | Cyclosporine, doxorubicin, paclitaxel, tacrolimus, |
| | sirolimus, fludarabine, ranpirnase |
| Tissue Absorption | Fish oil, squid oil, omega 3 fatty acids, vegetable oils, |
| Enhancers | lipophilic and hydrophilic solutions suitable for enhancing |
| | medication tissue absorption, distribution and permeation |
| Anti-Adhesion Agents | Hyalonic acid, human plasma derived surgical |
| | sealants, and agents comprised of hyaluronate and |
| | carboxymethylcellulose that are combined with |
| | dimethylaminopropyl, ehtylcarbodimide, hydrochloride, |
| | PLA, PLGA |
| Ribonucleases | Ranpirnase |
| Germicides | Betadine, iodine, sliver nitrate, furan derivatives, |
| | nitrofurazone, benzalkonium chloride, benzoic acid, |
| | salicylic acid, hypochlorites, peroxides, thiosulfates, |
| | salicylanilide |
| Antiseptics | Selenium |

In addition to or in conjunction with the above table, the medical agent of the present invention can further include an antimicrobial agent. As utilized herein, the term antimicrobial agent shall include antibiotic, antimicrobial, antibacterial, germicidal agents and the like. There may be a combination of antimicrobial agents. In addition, example antibiotics which may be used in conjunction with the present invention include: aminoglycosides, such as gentamicin, kanamycin, neomycin, paromomycin, streptomycin, or tobramycin; ansamycins, such as rifamycin, or rifampin; cephalosporins, such as cephalexin, cephaloridine, cephalothin, cefazolin, cephapirin, cephradine, or cephaloglycin; chloramphenicols; macrolides, such as erythromycin, tylosin, oleandomycin, or spiramycin; penicillins, such as penicillin G and V, phenethicillin, methicillin, oxacillin, cloxacillin, dicloxacillin, floxacillin, nafcillin, ampicillin, amoxicillin, or carbenicillin; suflonamides; tetracyclines, such as tetracycline, oxytetracycline, chlortetracycline, methacycline, demeclocycline, rolitetracycline, doxycycline, or minocycline; trimethoprim-sulfamethoxazole; polypeptides, such as bacitracin, polymyxins, tyrothricin, or vancomycin; and miscellaneous antibiotics, such as lincomycin, clindamycin, or spectinomycin, in addition to oxytetracycline hydrochloride (OTC).

There are a plurality of germicides which may at least partially form the medical agent of the present invention, including phenols; cresols; resorcinols; substituted phenols; aldehydes; benzoic acid; salicyclic acid; iodine; iodophors, such as betadine; chlorophors, such as hypochlorites; peroxides; such as hydrogen peroxide and zinc peroxide; heavy metals and their salts, such as merbromin, silver nitrate, zinc sulfate; surface-active agents, such as benzalkonium chloride; furan derivatives, such as nitrofurazone; sulfur and thiosulfates; salicylanilides; and carbanilides.

The amount of the antibiotic, bactericidal, or germicide present in an application of a marking varies with the nature of antibiotics or germicides employed and to some extent the method applying the marking as understood by one of ordinary skill in the art.

FIGS. 7A, 7B, and 7C are perspective illustrations of the medicated ink marker 60 in the form of a rounded medicated ink marker 60a, a squared medicated ink marker 60b, and a pointed medicated ink marker 60c, in accordance with embodiments of the present invention. The embodiments illustrated, as well as equivalents as understood by one of ordinary skill in the art, are referred to herein with the general reference of the medicated ink marker 60. However, the present invention is not limited to the embodiments illustrated, but rather anticipates other shapes and forms of the medicated ink marker 60 that can perform the stated functions as described herein.

The medicated ink marker 60 is saturated with the drug or agent to an extent such that a predetermined dosage amount of the drug or agent is held within the medicated ink marker 60. As the medicated ink marker 60 makes contact with the surface 12 a wicking action draws the drug or agent from the medicated ink marker 60 to the surface 12. Once the drug or agent contained within the medicated ink marker 60 has wicked to the surface 12, the complete dosage indicated on the medicated ink marker 60 has been delivered. The entire dosage of the drug or agent is contained within the porous medicated ink marker 60. There is no reservoir connected with the porous medicated ink marker 60 from which the medicated ink marker 60 can draw any drug or agent. Accordingly, once the medicated ink marker 60 is utilized on the desired surface 12, the medicated ink marker 60 is not reused and is disposed of by the user. In other words, the medicated ink marker 60 is unlike a pen or highlighter, that draws liquid from a reservoir and can thus be readily reused. The medicated ink marker 60 is intended to be a one-time use device that contains a metered dose of drug or agent that will cease to emit from the marker when the complete dose has been administered.

The rounded medicated ink marker 60a has at least one end that is generally rounded, and otherwise has a cylindrical cross-section. A rounded portion 64 of the medicated ink marker 60a is intended to make contact with the surface 12 upon which the drug or agent is applied. As such, where the rounded portion 64 of the medicated ink marker 60a makes contact, the wicking action occurs and the drug or agent wicks to the surface 12 where desired. When the drug or agent stops wicking from the medicated ink marker 60a, the complete dose of the drug or agent has been delivered.

As with the rounded medicated ink marker 60a, the squared medicated ink marker 60b, and the pointed medicated ink marker 60c likewise have portions intended to make contact with the surface 12 upon which the drug or agent is to be applied. The squared medicated ink marker 60b combines a flat surface 66 and an edge surface 68, each of which can be used in applying the drug or agent to the surface 12. The pointed medicated ink marker 60c has a pointed portion 70 and a side surface 72 that can each be utilized in applying the drug or agent to the surface 12. As is understood by one of ordinary skill in the art, the different shapes of contact surfaces illustrated herein, in addition to other possible shapes, can each result in a different marking 14 resulting. For example, the pointed portion 70 can be utilized to form a small point, or to form a thin line on the surface 12. The flat surface 66 can be used to form a relatively thicker line. The rounded portion 64 can be used to form an even thicker line. Accordingly, the present invention is not limited to the specific embodiments shown in the figures. Rather, the present invention anticipates that a number of different shapes can be utilized in forming the medicated ink marker 60, and each shape can result in a different shaped or sized marking 14.

The medicated ink marker 60 is formed of a generally porous material, such as a plastic, composite, rubber, rubberized plastic or composite, porous synthetic, and the like. As discussed above, the material of the medicated ink marker 60 forms a wick that maintains wicking characteristics. By wicking characteristics, what is meant is that although porous, the material forming the medicated ink marker 60 is configured to create capillary action to draw liquid from one end to the other of the material. When the medicated ink marker 60 makes contact with the surface, the capillary action initiates, and the fluid contained within the porous material wicks out to the surface 12.

The medicated ink marker 60 containing the ink can be used to apply the marking 14 to the surface 12. The clinical user draws the desired marking 14 directly on the surface 12 with the ink containing one or more therapeutic agents. Different color medicated ink markers 60 can contain different medication classifications or types of medication based on different color schemes. The medicated ink marker 60 can also be utilized in forming simple color patterns, symbols, or text.

The medicated ink marker 60 can fit within a holder 74 for holding the porous applicator, an example embodiment of which is shown in FIG. 8. The holder 74 has a coupling 76 for receiving the porous applicator, the specific mechanism of which can vary as understood by one of ordinary skill in the art, and can include adhesive, mechanical fastener, and the like. The holder 74 is a structure that is more easily manipulated by the user when applying the medicated ink marker 60 against the surface 12. The holder 74 represents any number of different variations of tools or implements for holding the medicated ink marker 60 while the drug or agent is applied to the surface 12. Such tools or implements can include other variations of handles or grips, as well as other elongate structures such as tongs, clamps, shafts, surgical tools, and the like, or other structures that serve the function of providing something for the user to grasp other than directly grasping the medicated ink marker 60 directly.

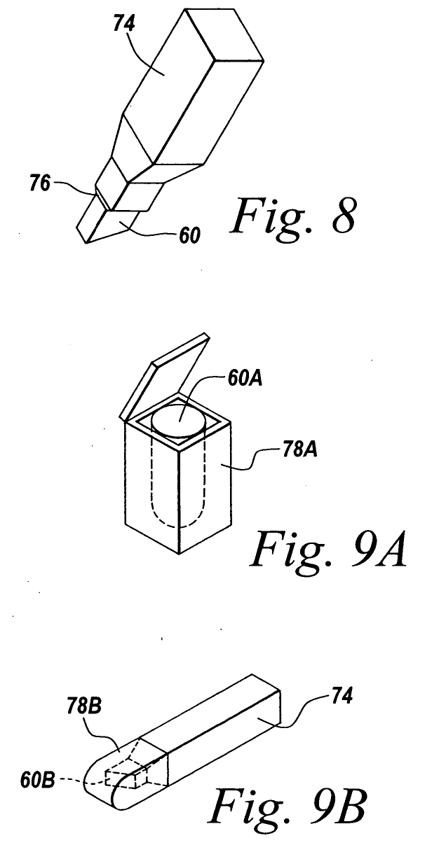

FIGS. 9A and 9B are perspective illustrations of a cap 78a and 78b that can be used in conjunction with the medicated ink marker 60 (shown as embodiments rounded medicated ink marker 60a and square medicated ink marker 60b) in accordance with the present invention. Because of the porous nature of the medicated ink marker 60, there is a substantial penetration of the medicated ink marker 60 by air in the surrounding atmosphere. Such air can be detrimental to the liquid containing the drug or agent within the medicated ink marker 60, and can result in the medicated ink marker 60 drying out prior to use. This is especially of concern when the medicated ink marker is loaded with the drug or agent at a manufacturing facility, and then shipped for later use. Upon loading the medicated ink marker 60 with the drug or agent the cap 78a and 78b can be placed on or over the medicated ink marker 60, or the medicated ink marker 60 can be placed in the cap 78a and 78b, to preserve the liquidity of the drug or agent solution contained within the porous structure of the medicated ink marker 60. The cap 78a and 78b provides a seal that substantially hinders the evaporation of the drug or agent from the medicated ink marker 60, thus preserving the functionality of the medicated ink marker 60 when stored for a period of time after the drug or agent is applied.

As is understood by one of ordinary skill in the art, the shape, size, and dimension of the cap 78a and 78b can vary depending at least in part on the shape, size, and dimension of the embodiment of medicated ink marker 60 that requires use of the cap 78a and 78b. As such, the figures show two example embodiments of the cap 78a and 78b, but the present invention is by no means limited to use with only those cap embodiments shown. It is anticipated that a number of different cap variations can perform the same functionality as those illustrated herein.

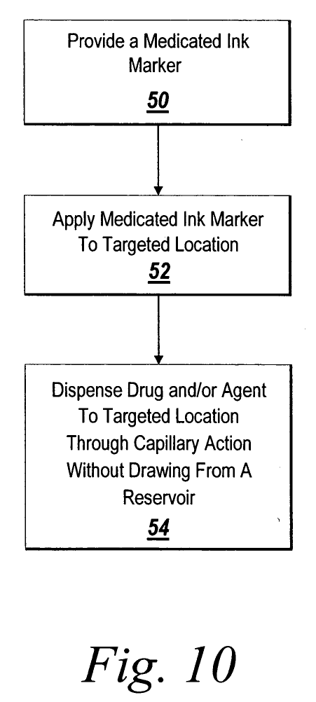

FIG. 10 illustrates an example method of applying a drug or agent to a targeted location. The medicated ink marker 60 is provided containing at least one drug or agent (step 50). The medicated ink marker 60 contains at least one drug or agent after being loaded with such drug or agent. The loading of the medicated ink marker 60 can occur, for example, at a manufacturing facility or on site by the user where the drug or agent is to be applied. Such application by the user could include spraying the medicated ink marker 60 with the drug or agent, dipping the medicated ink marker into the drug or agent, and the like.

As previously mentioned, the medicated ink marker 60 supports capillary action. A user directs the medicated ink marker 60 to contact the targeted location on the surface 12 (step 52). The contact between the surface 12 and the medicated ink marker 60 creates a capillary action dispensing the at least one drug or agent to the surface 12 without drawing additional medicated agent from another location (step 54).

If no additional markings 14 are required, the process is complete. If additional markings are required, the user can repeat the process. The user can apply different agents to the same area of surface 12 as needed or apply more of the same agents with subsequent marker applications to increase dosage. The present invention can provide multiple ink markings 14 with different therapeutic effects and independent activation and/or release rates on the surface 12 of the patient.

The markings 14 of the present invention enable the distribution of agents to a targeted location on a patient's body. The ink is relatively thin and unobtrusive to the applied surface. The marking 14 can further provide relevant information concerning the agents combined with the ink, as well as other characteristics of the ink and/or the agent, such as drug type, drug brand, drug dosage, dimensions, sizing, placement, orientation, and the like.

The present invention has many different therapeutic uses. More specifically, one clinical use for the marking 14 containing at least one agent is for application onto the surface 12. The surface 12 can include both internal and external sides of a patient's skin, as well as any other tissue within the patient. In some instances, the tissue may only be accessible during a surgical or other medical procedure.

All identifiable and/or detectable drug exuding inks that form the markings 14 can be made as a permanent marking or as a temporary marking, which can be absorbed by the local surface 12. More specifically, the marking 14 can have a relatively short term therapeutic effect, or the marking 14 can have a longer term, more permanent effect. A tattoo, for example, is representative of an ink that is a longer term application. Whereas, an ink that is applied and is absorbed in a matter of minutes or days has a shorter term therapeutic effect. Inks and agents combined with inks can have therapeutic effects ranging between the shorter term and longer term applications.

The present invention, thus, provides a medicated ink marker that is porous and supports a capillary action for delivery of a drug or agent to a desired surface, such as tissue or the surface of a medical device. The medicated ink marker does not contain a separate reservoir of liquid from which to draw. Rather, the medicated ink marker is formed of a porous material that serves as both the application point, and the drug or agent storage mechanism. An entire desired dosage of the drug or agent is contained within the porous medicated ink marker, thus a separate reservoir is not needed. As such, a user of the medicated ink marker can be assured that there is no possibility of applying more of a drug or agent than is provided in the dosage as marked on the outside of the medicated ink marker or on associated packaging. In other words, because there is no reservoir of drug or agent continually supplying the porous applicator, there is a limited amount of drug or agent contained within the porous medicated ink marker which corresponds to a desired dosage. The medicated ink marker is then disposed of after the drug or agent has been administered because there is no substantial drug or agent remaining in the medicated ink marker after application of the drug or agent dosage. Thus an efficient and accurate application of the drug or agent in the specified dosage amounts results from the use of the medicated ink marker of the present invention.

Numerous modifications and alternative embodiments of the present invention will be apparent to those skilled in the art in view of the foregoing description. Accordingly, this description is to be construed as illustrative only and is for the purpose of teaching those skilled in the art the best mode for carrying out the present invention. Details of the structure may vary substantially without departing from the spirit of the present invention, and exclusive use of all modifications that come within the scope of the appended claims is reserved. It is intended that the present invention be limited only to the extent required by the appended claims and the applicable rules of law.

1. A medicated ink marker, comprising:
a porous applicator; and
at least one medicated agent disposed within the porous applicator, wherein the at least one medicated agent is one of a non-antiseptic medicated agent and an antiseptic with an additional therapeutic function medicated agent;
wherein the porous applicator supports capillary action, such that contact of the applicator against a targeted location results in the at least one medicated agent dispensing from the porous applicator to topically apply the at least one medicated agent to the targeted location in a detectable manner without drawing additional medicated agent from another location.

2. The medicated ink marker of claim 1, further comprising a holder for holding the porous applicator, the holder having a coupling for receiving the porous applicator.

3. The medicated ink marker of claim 2, wherein the holder comprises an elongate structure.

4. The medicated ink marker of claim 2, wherein the porous applicator is removably and replaceably couplable with the holder.

5. The medicated ink marker of claim 2, wherein the porous applicator couples with the holder using a wick holder.

6. The medicated ink marker of claim 2, wherein the holder comprises a casing that does not contain a medicated ink reservoir.

7. The medicated ink marker of claim 2, wherein the holder serves as a grip for a user to grasp to control the porous applicator and application of the medicated agent to the targeted location.

8. The medicated ink marker of claim 2, further comprising an identifier placed on the holder to convey information concerning the at least one medicated agent.

9. The medicated ink marker of claim 8, wherein the identifier is at least one of color coded, text-based, symbol-based, and code-based.

10. The medicated ink marker of claim 1, wherein the porous applicator comprises a wick.

11. The medicated ink marker of claim 1, further comprising a removable cap placed over the porous applicator to hinder evaporation of the medicated agent.

12. The medicated ink marker of claim 11, wherein the removable cap is replaceable.

13. The medicated ink marker of claim 1, further comprising an identifier placed on the porous applicator to convey information concerning the at least one medicated agent

14. The medicated ink marker of claim 13, wherein the identifier is at least one of color coded, text-based, symbol-based, and code-based.

15. A method of applying at least one medicated agent to a targeted location, comprising:
providing a porous applicator containing at least one medicated agent, wherein the porous applicator supports capillary action and wherein the at least one medicated agent is one of non-antiseptic and antiseptic with an additional therapeutic function; and
a user topically applying the at least one medicated agent to the targeted location in a detectable manner by directing the porous applicator to contact the targeted location dispensing the at least one medicated agent from the porous applicator to topically apply the at least one medicated agent to the targeted location without drawing additional medicated agent from another location.

16. The method of claim 15, further comprising coupling the porous applicator to a holder, such that the user can control the porous applicator for application of the at least one medicated agent.

17. The method of claim 16, wherein the holder comprises an elongate structure.

18. The method of claim 16, wherein the porous applicator is removably and replaceably couplable with the holder.

19. The method of claim 16, wherein the porous applicator couples with the holder using a wick holder.

20. The method of claim 16, wherein the holder comprises a casing that does not contain a medicated ink reservoir.

21. The method of claim 16, wherein the holder serves as a grip for a user to grasp to control the porous applicator and application of the medicated agent to the targeted location.

22. The method of claim 16, further comprising an identifier placed on the holder to convey information concerning the at least one medicated agent.

23. The method of claim 22, wherein the identifier is at least one of color coded, text-based, symbol-based, and code-based.

24. The method of claim 15, wherein the porous applicator comprises a wick.

25. The method of claim 15, further comprising a removable cap placed over the porous applicator to hinder evaporation of the medicated agent.

26. The method of claim 25, wherein the removable cap is replaceable.

27. The method of claim 15, further comprising an identifier placed on the porous applicator to convey information concerning the at least one medicated agent

28. The method of claim 27, wherein the identifier is at least one of color coded, text-based, symbol-based, and code-based.
